(19) United States
(12) Patent Application Publication
(10) Pub. No.: US 2005/0020890 A1
(43) Pub. Date: Jan. 27, 2005

(54) Device for introducing into hollow organs of the body

(76) Inventor: Werner Schregel, Krefeld (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/492,278

(22) PCT Filed: Oct. 8, 2002
(86) PCT No.: PCT/EP02/11242

(30) Foreign Application Priority Data
Oct. 8, 2001 (DE) 201 16 460.4

(51) Int. Cl. A61B 5/00

(52) U.S. Cl. 600/309

(57) ABSTRACT

The invention relates to a device for introducing into hollow organs of the body, in particular into blood vessels. Said device consists of a substantially longitudinal instrument comprising a measuring device that is located in a closed channel in the vicinity of the device end that is introduced into the hollow organ of the body, in particular the blood vessel. The aim of the invention is to improve a device known per se and to widen its range of application. To achieve this, the device is provided with a measuring device that is suitable for measuring the composition and/or the characteristics of the blood, and/or a measuring device that is suitable for measuring the temperature of the blood flowing in the blood vessel and/or values that can be determined by temperature measurements, in particular the flow rate, and/or a measuring device for measuring haemodynamic parameters of the blood and/or values that can be determined by pressure measurements.

The invention relates to a device which can be introduced into hollow organs of the body, especially into blood vessels, comprised of a substantially elongated instrument with at least one measuring device located in a closed channel of the inserted end of the device in the region of the hollow organ of the body, especially the blood vessel.

Known devices are configured as catheters and are used to withdraw liquids from hollow organs of the body, for example, blood vessels, as samples or to inject liquid into such hollow organs. For introduction into the hollow organ of the body, special instruments are required since the catheter, because of its flexible configuration, is not for example suitable for penetration of the tissue layers. The measuring device can be in part provided in the catheter. By means of such known measuring device, quantities of air in liquids, like for example in blood, can be detected. Should the operator for example require more detailed information as to the composition of the blood or as to the detected air quantity, the detected gas or the blood must be drawn off. The operator also cannot obtain any further information from the aforedescribed device. If the operator for example requires more detailed information as to the blood pressure for example, he must utilize additional instruments which in part may have to be additionally introduced into the body of the patient.

It is the object of the invention therefore to improve the aforedescribed device and make the same more versatile.

This object is achieved in that, for the measurement of the composition and/or the properties of the blood, a suitable measuring device is provided. By means of such a measuring device, body-specific laboratory values, for example, of hemoglobin, oxygen saturation, electrolytes, blood gases and viscosity can be obtained. It is also possible to determine the concentration of gases or chemical substances like, for example, medicaments or dye indicators, so that should levels fall below certain thresholds, the deficient substances can be supplied to the treated patients in the optimally determined dosages. Since the measuring devices are inserted into a closed-channel, they do not come into contact with the medium which is to be explored, for example, the blood, and to that extent they are reusable. After a use, the measuring device can be withdrawn without difficulty from the insertion instrument or the channel and simply inserted into a new device.

The measuring device can be configured as an optical measurement device and the instrument can at least in the region of the measuring device be made transparent to the optical radiation required for the measurement and can thus be comprised of a material enabling an optical measurement. For that purpose, the instrument can for example have a window which enables an optical detection therefrom. It is for example also possible to make the entire instrument of an optically transparent material.

Alternatively, the instrument in the measurement region can have a semipermeable membrane. Such a membrane indeed allows passing of ions for investigation of body-specific laboratory values; however a penetration of blood, protein, infectious substances or other materials is precluded so that the measuring device also remains reusable.

Conceivable are for example measuring devices comprised of a transmitter and a receiver and which, based upon the refractive index of transmitted and received light beams can provide information as to the material to be investigated between the transmitter and receiver. Optical measurement devices are also conceivable in which light beams of different wavelengths are emitted and at least partially reflected so that information as to substances found in the blood can be provided. Appropriate spectroscopic measuring devices are also conceivable. The measuring device can be integrated in a chip, formed as a chip or connected therewith.

In another embodiment of the invention, for measurement of the temperature of the blood flowing in the blood vessel and/or of values determinable from temperature measurements, especially the flow velocity, a suitable measuring instrument is provided. The instrument is comprised at least in the measuring region of a material which enables a temperature measurement to be made. It will be self-understood that this measuring device together with a measuring device for measuring the composition and/or to characteristics of the blood can be provided in a device according to the invention. Since the measuring device can be inserted into a closed end channel of the instrument, it cannot come into contact with the medium to be investigated, like for example, blood and is to that extent reusable. After use, the measuring device can be withdrawn from the insertion instrument without difficulty and can be simply inserted into a new instrument.

To this end the measuring device can be configured as a temperature measurement device and/or as a heat-flow sensor.

Insofar as, for example, the flow velocity is to be measured, the temperature measuring device can be comprised for example of spaced-apart temperature sensors, whereby one temperature sensor is spaced by an additional insulating material from the medium to be measured. The measured difference enables conclusions as to the flow velocity which will supply information as to the blood supply to the organ. A volume can also be determined that for example is displaced within a given time interval by the heart. As the temperature measuring device, a thermal element [thermal couple] can for example be used.

The measuring device can also be integrated in a chip, configured as a chip or connected with the latter.

In a further embodiment of the invention, hemodynamic parameters of the blood can be measured and/or from a pressure measurement or from pressure measurements given values can be obtained from a suitable measuring device. This measuring device can together with a measuring device for measuring the composition and/or the characteristics of the blood and/or a measuring device for measuring the temperature and/or for determining certain values from the temperature measurement, can be provided in the instrument according to the invention. Since the measuring device is enclosed in a closed end channel, it does not come into contact with the medium to be investigated, like, for example, blood and to that extent is replaceable. After use the measuring device can be withdrawn from the instrument without difficulty and then inserted into a new instrument in a simple manner.

Under hemodynamic parameters, for example blood pressure, heart rhythm or heart minute volume flow can be understood. Such a measuring device can also determine particles found in blood embolisms, blood coagulants, bone splinters, bone cements or fats. Simultaneously measurements can be made which can provide information as to the flow velocity and the heart minute volume.

The measuring device can be configured for measurement of pressure, especially the reflected sonic pressure of a sound-wave generator and the instrument can, at least in the region of the measuring device, have a pressure-transmitting region.

The pressure-measuring device can be configured as a pressure pickup, especially a piezo element. There are however also for example, pressure pickups operating on a magnetic basis or pressure pickups which can be configured as microphones.

The pressure-measuring device can also be configured as an ultrasonic probe. In that case it is possible to provide a plurality of such measuring devices in longitudinally-spaced relationship to one another. In this case one can use the so-called Doppler effect in which the frequency change between the waves outputted by the receiver and captured by the receiver is evaluated.

The measuring device can also be integrated in a chip, configured as a chip or connected therewith.

The data measured by and obtained from the respective measuring device and/or the chip can be supplied in different ways to the control and display unit located externally of the instrument so that the user can acquire the data which is relevant for him. In the simplest embodiment this is achieved by means of a cable connection. The transmission by radio waves or infrared is also conceivable since then the data can additionally be archived. It is also possible, to the extent that the data does not simultaneously have to be read, to call up this data first after the processing and then analyze it.

So that the instrument according to the invention also can be used to stabilize heart frequency during operations the instrument is provided, in addition, with a pulse generator, preferably a heart pacemaker, and a pulse outputting device can be provided. In this case as well, the device can be integrated in a chip, configured as a chip or connected with a chip.

In addition, at least one infusion or suctioning passage can be provided which is effective at the end region in a blood vessel through an opening. This has the advantage that using the instrument, liquid can be drawn off or medicaments or solutions injected into the hollow organ of the body which is relevant.

It has been found to be advantageous to form the device as rigid and with a tip which can be inserted into the hollow organ of the body. In such an embodiment, the instrument according to the invention will be suitable for penetration of tissue layers like, for example, the skin so that no separate instrument is required for inserting the device according to the invention into the hollow organ of the body to be investigated.

The closed end channel or a further channel of closed configuration can be used to therapeutically influence the temperature, for example by introducing a heating element and/or cooling element or by introducing a temperature control medium which can especially be circulated. The cooling element can be brought to the desired temperature for example prior to insertion. Advantageously a temperature control medium is circulated in the temperature control element so that the temperature control medium is continuously maintained at the desired temperature by a corresponding temperature controller. It is however also possible to conduct the temperature control medium directly through the passage. In this case, preferably a partition extends along the passage within the latter. The partition has advantageously in the region of the closed end, a cutout so that the channel has a generally U-shaped path for the temperature control medium and through which the latter, for example a hot or cold common salt containing solution, can circulate.

No detailed explanation is required to show that the instrument according to the invention is suitable both for venous and arterial use. It will be self-understood that the instrument according to the invention can also be used for investigations of the heart.

In the following an embodiment of the invention is described which is illustrated in the drawings. They show:

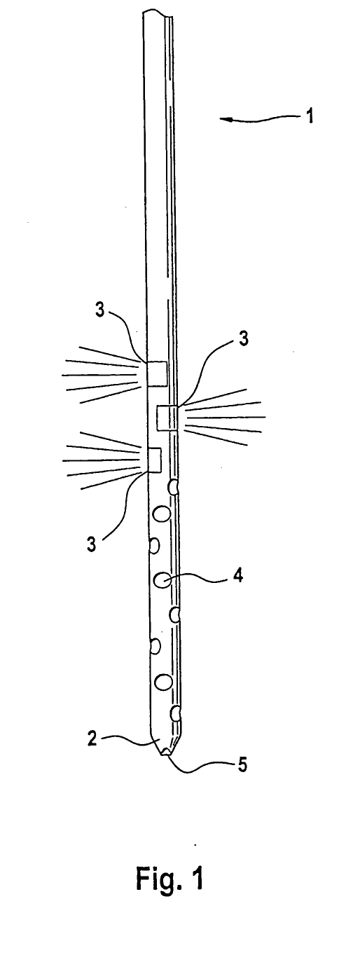

FIG. 1 a side view of a device according to the invention,

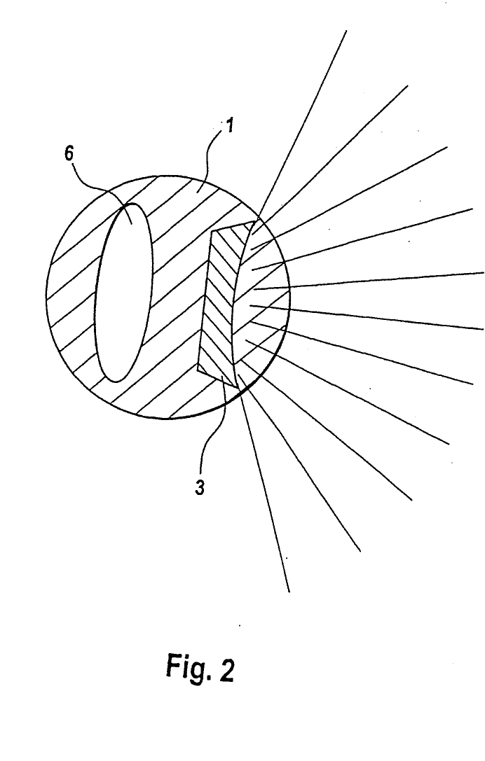

FIG. 2 a section through a device with an infusion or evacuation passage and at least a measuring device integrated therein,

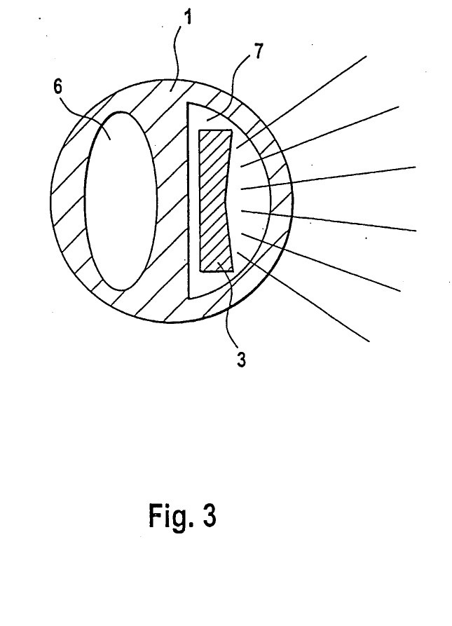

FIG. 3 a section through a device with an infusion or evacuation passage and a channel closed at an end in which at least one reusable measuring device is inserted, and

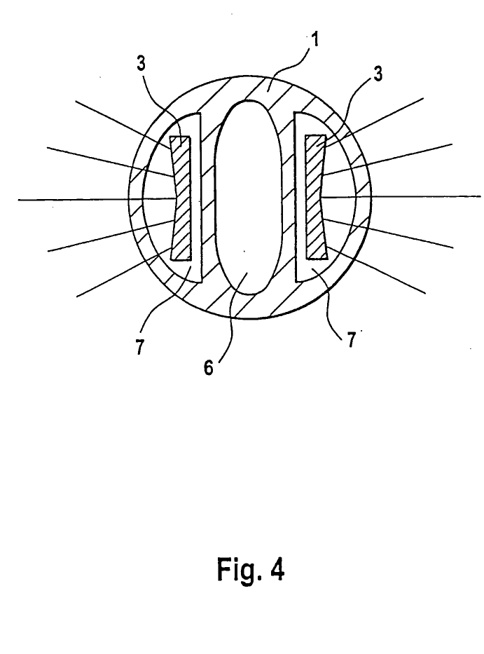

FIG. 4 a section through a device with an infusion or suction passage and two channels closed at their ends, each of which has at least one measuring device inserted therein.

In all of the Figures for the same or similar components, the same reference characters have been used.

FIG. 1 shows a device 1 which is comprised of a substantially tubular instrument. In that illustrated embodiment, the instrument 1 is in the form of a catheter which can be inserted in a blood vessel (not shown), whereby one end 2 with the instrument 1 is of frustoconical shape for insertion into a blood vessel.

Spaced from the end 2 of the device 1, measuring units 3 are provided. The measuring units 3 are preferably so arranged that the radiation angles to the extent provided for measurement of gas concentrations and/or medicament concentrations, overlap each other so that the entire vessel volume of the blood vessel can be monitored.

In the illustrated embodiment a total of three measuring devices are to be found in the instrument 1, whereby advantageously one measuring device serves for the measurement of the composition and/or characteristics of the blood, one measuring device serves for measuring the temperature and/or values which can be determined from such temperature measurements, and one measuring device serves for the measurement of hemodynamic parameters and/or values which can be determined from pressure measurement.

It is thus possible, overall, for one operator during operations to determine important parameters and values by means of a single instrument 1 which can be located for example in a blood vessel.

Since the instrument 1 is simultaneously configured as a catheter, between the measuring devices 3 and the end 2 of the instrument 1, lateral openings 4 as well as, in the region of the end 2, end openings 5 are provided which are connected as has been shown in FIG. 2 with an infusion passage or suction passage 6.

The other end of the instrument 1 is connected with a commercially available but not illustrated infusion or suction device for the infusion or suction passage 6 as well as to a control and display unit for the measuring devices 3 which also have not been illustrated.

The instrument 1 is prior to operation inserted into a blood vessel which has not been shown and placed at the desired location from which the measurements are to be taken. Through the openings 4 or 5, substances like for example medicaments, can be injected or sampled drawn off by suction while simultaneously the measuring devices allow measurements to be taken.

FIG. 2 shows a variant of the instrument 1 in which the measuring device 3 is fixed and integrated in the instrument 1. The measuring device 3 shown by way of example in FIG. 2 is comprised of a receiver and a transmitter that are oriented at an angle to one another. The waves radiated from the transmitter are reflected from the liquid and air bubbles which may possible be found therein, for example, and are again picked up by the receiver. Differences arise depending upon the reflected medium, that is liquid or air bubbles, in the frequency of the transmitted and received waves so that means of this technique, for example, particles which can be found in blood, like for example, bone splinters, can be detected.

FIG. 3 shows an embodiment of the invention in which, in the instrument 1, the measurement unit 3 is located in a closed-end channel 7. Since the channel 7 is closed at its end, the measuring units 3 located and shiftable therein, do not come into contact with the liquid to be monitored and flowing around the instrument 1. Following the operation, the measuring devices 3 are withdrawn from the channel (7) of the instrument 1 and can be inserted in a new instrument and thus reused without requiring an expensive sterilization for them. Also here, the infusion passage or suction passage 6 is located in the other half of the instrument.

Depending upon the kind of operation and the parameters to be monitored, the instrument 1 can have a variety of measuring devices located in the channel 7 so that the resulting instrument 1 can be used optimally to match the needs of any special application.

In the embodiment 4 of the instrument 1 illustrated in FIG. 4, the infusion passage or suction passage 6 is centrally located whereby passages 7 closed at their ends are provided on both sides of the infusion or suction passage 6. In the passages 7, measuring devices 3 are inserted. Further explanations to the effect that a plurality of measuring devices 3 are provided in each channel 7 is not necessary.

In addition, a pulse-generating and outputting device can be provided in the channel 7. This can serve for example during heart operations to stabilize the heart rate.

The instrument 1 shown in FIGS. 1 and 4 is a catheter. It will be self-understood that the instrument 1 can also be configured as a needle whereby in this case, the end 2 can have a point. With such an embodiment the instrument 1 is suitable for penetrating a tissue.

After penetrating the tissue layers, for example, a medicament can be supplied in metered amounts while simultaneously the medicament concentration in the relevant organ of the body can be determined.

To the extent that the instrument 1 is configured as a needle, there need not be any lateral opening 4.

1. A device (1) for insertion into a hollow organ of the body, especially into a blood vessel, comprised of a substantially elongated instrument with at least one end (2) insertable into a region of the hollow organ of the body, especially the blood vessel in which there is a closed channel receiving a closed channel (7) receiving a measuring device (3) characterized in that for the measurement of the composition and/or the characteristics of the blood, a suitable measuring device (3) is provided.

2. The device (1) according to claim 1 characterized in that the measuring device (3) is configured as an optical measuring device (3) and that the instrument at least in the region of the measuring device (3) is transparent for the optical radiation for the measurement and is comprised of a material enabling an optical measurement.

3. The device (1) according to claim 1 characterized in that the measuring device (3) is integrated in a chip.

4. The device (1) for insertion in a hollow organ of a body, especially in a blood vessel, comprised of a substantially elongated instrument with at least one end (2) insertable into the region of the hollow organ of the body, especially the blood vessel in which the device (1) has a closed channel (7) in which a measuring device (3) is found, especially in accordance with claim 1, characterized in that an appropriate measuring device (3) is provided for measuring the temperature of the blood flowing and the blood vessel and/or values determinable from the temperature measurement, especially the flow velocity.

5. The device (1) according to claim 4 characterized in that the measuring device (3) is configured as a temperature measuring device and/or as a heart flow sensor.

6. The device (1) according to claim 4, characterized in that the measuring device (3) is integrated in a chip.

7. The device (1) for insertion in a hollow organ of a body, especially in a blood vessel, comprised of a substantially elongated instrument with at least one measuring device elongated in a closed channel (7) of the end (2) of the instrument disposed in the region of the hollow organ of the body, especially in the blood vessel, especially according to claim 1, characterized in that for the measurement of hemodynamic parameters of the blood and/or of values which can be determined from pressure measurements, a suitable measuring device (2) is provided.

8. The device (1) according to claim 7 characterized in that the measuring device (3) is configured for the measurement of pressure, especially reflected sound pressure of a sound generator and the instrument at least in the region of the measuring device (3) has a pressure-transmitting region.

9. The device (1) according to claim 8 characterized in that the pressure measurement device is configured as a pressure pickup, especially a piezo element.

10. The device (1) according to claim 9 characterized in that the pressure measurement device is configured as an ultrasonic probe.

11. The device (1) according to claim 7, characterized in that the measuring device (3) is integrated in a chip.

12. The device (1) according to claim 1, characterized in that in the instrument, additionally a pulse-generating and transmitting device is provided, especially for heart pace-maker pulses.

13. The device (1) according to claim 12 characterized in that the device is integrated in a chip.

14. The device (1) according to claim 1, characterized in that additionally at least one infusion or suction passage (6) which has at least one opening (5) in the end region located in the blood vessel, is provided.

15. The device (1) according to claim 1, characterized in that the device (1) is rigid and has at its end insertable into the hollow organ of the body, a point (2).

16. The device (1) according to claim 1, characterized in that the closed end channel or a further closed channel is provided for therapeutic influence upon the temperature, for example by means of introducing hot and/or cold elements or by delivering an especially circulating temperature-control medium.
